SEQUENCE LISTING (35 sequences)

SEQ ID NO: 1 - protein, 8 aa, Artificial Sequence
  FLAG epitope
DYKDDDDK

SEQ ID NO: 2 - protein, 15 aa, Artificial Sequence
  AVITAG epitope
GLNDIFEAQKIEWHE

SEQ ID NO: 3 - protein, 17 aa, Artificial Sequence
  Rev peptide
TRQARRNRRRRWRERQR

SEQ ID NO: 4 - protein, 12 aa, Artificial Sequence
  Tat peptide
GRKKRRQRRRPQ

SEQ ID NO: 5 - DNA, 40 bp, Artificial Sequence
  6H7 aptamer
gctatgggtggtctggttgggattggccccgggagctggc

SEQ ID NO: 6 - protein, 28 aa, Artificial Sequence
  residues 13-166 of the M1 protein
TTEAAFGLVCATCEQIADSQHRSHRQMA

SEQ ID NO: 7 - protein, 5 aa, Artificial Sequence
  enterokinase cleavage sequence
DDDDK

SEQ ID NO: 8 - protein, 16 aa, Artificial Sequence
  peptide
GDSTRTGRTGHHHHHH

SEQ ID NO: 9 - protein, 30 aa, Artificial Sequence
  M2e peptide
SLLTEVETPIRNEWGCRCNDSSDPHHHHHH

SEQ ID NO: 10 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 11 - protein, 130 aa, Artificial Sequence
  MS2 capsid mutant T16C
MASNFTQFVLVDNGGCGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 12 - protein, 259 aa, Artificial Sequence
  MS2 capsid dimer mutant T16C, T145C
MASNFTQFVLVDNGGCGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYASNFTQFVLVDNGGCGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 13 - protein, 130 aa, Artificial Sequence
  MS2 capsid mutant T16C, C47A, C102A
MASNFTQFVLVDNGGCGDVTVAPSNFANGVAEWISSNSRSQAYKVTASVRQSSAQNRKYTIKVEVPKVATQTVGGVELPVAAWRSYLNMELTIPIFATNSDAELIVKAMQGLLKDGNPIPSAIAANSGIY

SEQ ID NO: 14 - protein, 130 aa, Artificial Sequence
  MS2 capsid mutant T16C, C47S, C102S
MASNFTQFVLVDNGGCGDVTVAPSNFANGVAEWISSNSRSQAYKVTSSVRQSSAQNRKYTIKVEVPKVATQTVGGVELPVAAWRSYLNMELTIPIFATNSDSELIVKAMQGLLKDGNPIPSAIAANSGIY

SEQ ID NO: 15 - protein, 188 aa, Physalis mottle virus
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 16 - protein, 188 aa, Artificial Sequence
  Physalis mottle virus coat protein mutant N25C
MDSSEVVKVKQASIPAPGSILSQPCTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 17 - protein, 188 aa, Artificial Sequence
  Physalis mottle virus coat protein mutant T26C
MDSSEVVKVKQASIPAPGSILSQPNCEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 18 - protein, 188 aa, Artificial Sequence
  Physalis mottle virus coat protein mutant N25C, C75A
MDSSEVVKVKQASIPAPGSILSQPCTEQSPAIVLPFQFEATTFGTAETAAQVSLQTADPITKLTAPYRHAQIVEAKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 19 - protein, 188 aa, Artificial Sequence
  Physalis mottle virus coat protein mutant T26C, C75A
MDSSEVVKVKQASIPAPGSILSQPNCEQSPAIVLPFQFEATTFGTAETAAQVSLQTADPITKLTAPYRHAQIVEAKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 20 - protein, 161 aa, Microbacterium arborescens
MTDTNITTPALTADPEVAAAAAQFLTPVVHKMQALVVNGKQAHWNVRGSNFIAIHELLDSVVAHAQDYADTAAERIVALGLPIDSRVSTMAEKTSTAVPAGFAQWQDEIKAIVSDIDAALVDLQAAIDGLDEVDLTSQDVAIEIKRGVDKDRWFLLAHLAE

SEQ ID NO: 21 - protein, 108 aa, Bacteriophage HK97
MAIDVLDVISLSLFKQQIEFEEDDRDELITLYAQAAFDYCMRWCDEPAWKVAADIPAAVKGAVLLVFADMFEHRTAQSEVQLYENAAAERMMFIHRNWRGKAESEEGS

SEQ ID NO: 22 - protein, 108 aa, Artificial Sequence
  Bacteriophage HK97 gp6 connector protein C40A, C44A, K50C
MAIDVLDVISLSLFKQQIEFEEDDRDELITLYAQAAFDYAMRWADEPAWCVAADIPAAVKGAVLLVFADMFEHRTAQSEVQLYENAAAERMMFIHRNWRGKAESEEGS

SEQ ID NO: 23 - protein, 108 aa, Artificial Sequence
  Bacteriophage HK97 gp6 connector protein C40A, C44A, N97C
MAIDVLDVISLSLFKQQIEFEEDDRDELITLYAQAAFDYAMRWADEPAWKVAADIPAAVKGAVLLVFADMFEHRTAQSEVQLYENAAAERMMFIHRCWRGKAESEEGS

SEQ ID NO: 24 - protein, 108 aa, Artificial Sequence
  Bacteriophage HK97 gp6 connector protein K50C
MAIDVLDVISLSLFKQQIEFEEDDRDELITLYAQAAFDYCMRWCDEPAWCVAADIPAAVKGAVLLVFADMFEHRTAQSEVQLYENAAAERMMFIHRNWRGKAESEEGS

SEQ ID NO: 25 - protein, 108 aa, Artificial Sequence
  Bacteriophage HK97 gp6 connector protein N97C
MAIDVLDVISLSLFKQQIEFEEDDRDELITLYAQAAFDYCMRWCDEPAWKVAADIPAAVKGAVLLVFADMFEHRTAQSEVQLYENAAAERMMFIHRCWRGKAESEEGS

SEQ ID NO: 26 - protein, 253 aa, Bacteriophage SPP1
MNIYDILDKVFTMMYDGQDLTDYFLVQEVRGRSVYSIEMGKRTIAGVDGGVITTESLPARELEVDAIVFGDGTETDLRRRIEYLNFLLHRDTDVPITFSDEPSRTYYGRYEFATEGDEKGGFHKVTLNFYCQDPLKYGPEVTTDVTTASTPVKNTGLAVTNPTIRCVFSTSATEYEMQLLDGSTVVKFLKVKYGFNTGDTLVIDCHERSVTLNGQDIMPALLIQSDWIQLKPQVNTYLKATQPSTIVFTEKFL

SEQ ID NO: 27 - protein, 30 aa, Artificial Sequence; identical to SEQ ID NO: 9
  ectodomain of influenza M2 protein

SEQ ID NO: 28 - protein, 24 aa, Artificial Sequence
  ectodomain of influenza M2 protein
SLLTEVETPIRNEWGCRCNDSSDP

SEQ ID NO: 29 - protein, 546 aa, Artificial Sequence
  histidine-tagged H5N1
METDTLLLWVLLLWVPGSTGDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKRGKPIPNPLLGLDSTRTGRTGHHHHHH

SEQ ID NO: 30 - protein, 3430 aa, West Nile virus
MSKKPGGPGKNRAVNMLKRGMPRGLSLIGLKRAMLSLIDGKGPIRFVLALLAFFRFTAIAPTRAVLDRWRGVNKQTAMKHLLSFKKELGTLTSAINRRSTKQKKRGGTAGFTILLGLIACAGAVTLSNFQGKVMMTVNATDVTDVITIPTAAGKNLCIVRAMDVGYLCEDTITYECPVLAAGNDPEDIDCWCTKSSVYVRYGRCTKTRHSRRSRRSLTVQTHGESTLANKKGAWLDSTKATRYLVKTESWILRNPGYALVAAVIGWMLGSNTMQRVVFAILLLLVAPAYSFNCLGMSNRDFLEGVSGATWVDLVLEGDSCVTIMSKDKPTIDVKMMNMEAANLADVRSYCYLASVSDLSTRAACPTMGEAHNEKRADPAFVCKQGVVDRGWGNGCGLFGKGSIDTCAKFACTTKATGWIIQKENIKYEVAIFVHGPTTVESHGKIGATQAGRFSITPSAPSYTLKLGEYGEVTVDCEPRSGIDTSAYYVMSVGEKSFLVHREWFMDLNLPWSSAGSTTWRNRETLMEFEEPHATKQSVVALGSQEGALHQALAGAIPVEFSSNTVKLTSGHLKCRVKMEKLQLKGTTYGVCSKAFKFARTPADTGHGTVVLELQYTGTDGPCKVPISSVASLNDLTPVGRLVTVNPFVSVATANSKVLIELEPPFGDSYIVVGRGEQQINHHWHKSGSSIGKAFTTTLRGAQRLAALGDTAWDFGSVGGVFTSVGKAIHQVFGGAFRSLFGGMSWITQGLLGALLLWMGINARDRSIAMTFLAVGGVLLFLSVNVHADTGCAIDIGRQELRCGSGVFIHNDVEAWMDRYKFYPETPQGLAKIIQKAHAEGVCGLRSVSRLEHQMWEAIKDELNTLLKENGVDLSVVVEKQNGMYKAAPKRLAATTEKLEMGWKAWGKSIIFAPELANNTFVIDGPETEECPTANRAWNSMEVEDFGFGLTSTRMFLRIRETNTTECDSKIIGTAVKNNMAVHSDLSYWIESGLNDTWKLERAVLGEVKSCTWPETHTLWGDGVLESDLIIPITLAGPRSNHNRRPGYKTQNQGPWDEGRVEIDFDYCPGTTVTISDSCEHRGPAARTTTESGKLITDWCCRSCTLPPLRFQTENGCWYGMEIRPTRHDEKTLVQSRVNAYNADMIDPFQLGLMVVFLATQEVLRKRWTAKISIPAIMLALLVLVFGGITYTDVLRYVILVGAAFAEANSGGDVVHLALMATFKIQPVFLVASFLKARWTNQESILLMLAAAFFQMAYYDAKNVLSWEVPDVLNSLSVAWMILRAISFTNTSNVVVPLLALLTPGLKCLNLDVYRILLLMVGVGSLIKEKRSSAAKKKGACLICLALASTGVFNPMILAAGLMACDPNRKRGWPATEVMTAVGLMFAIVGGLAELDIDSMAIPMTIAGLMFAAFVISGKSTDMWIERTADITWESDAEITGSSERVDVRLDDDGNFQLMNDPGAPWKIWMLRMACLAISAYTPWAILPSVIGFWITLQYTKRGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGHDEVQMIVVEPGKNVKNVQTKPGVFKTPEGEIGAVTLDYPTGTSGSPIVDKNGDVIGLYGNGVIMPNGSYISAIVQGERMEEPAPAGFEPEMLRKKQITVLDLHPGAGKTRKILPQIIKEAINKRLRTAVLAPTRVVAAEMSEALRGLPIRYQTSAVHREHSGNEIVDVMCHATLTHRLMSPHRVPNYNLFIMDEAHFTDPASIAARGYIATKVELGEAAAIFMTATPPGTSDPFPESNAPISDMQTEIPDRAWNTGYEWITEYVGKTVWFVPSVKMGNEIALCLQRAGKKVIQLNRKSYETEYPKCKNDDWDFVITTDISEMGANFKASRVIDSRKSVKPTIIEEGDGRVILGEPSAITAASAAQRRGRIGRNPSQVGDEYCYGGHTNEDDSNFAHWTEARIMLDNINMPNGLVAQLYQPEREKVYTMDGEYRLRGEERKNFLEFLRTADLPVWLAYKVAAAGISYHDRKWCFDGPRTNTILEDNNEVEVITKLGERKILRPRWADARVYSDHQALKSFKDFASGKRSQIGLVEVLGRMPEHFMVKTWEALDTMYVVATAEKGGRAHRMALEELPDALQTIVLIALLSVMSLGVFFLLMQRKGIGKIGLGGVILGAATFFCWMAEVPGTKIAGMLLLSLLLMIVLIPEPEKQRSQTDNQLAVFLICVLTLVGAVAANEMGWLDKTKNDIGSLLGHRPEARETTLGVESFLLDLRPATAWSLYAVTTAVLTPLLKHLITSDYINTSLTSINVQASALFTLARGFPFVDVGVSALLLAVGCWGQVTLTVTVTAAALLFCHYAYMVPGWQAEAMRSAQRRTAAGIMKNVVVDGIVATDVPELERTTPVMQKKVGQIILILVSMAAVVVNPSVRTVREAGILTTAAAVTLWENGASSVWNATTAIGLCHIMRGGWLSCLSIMWTLIKNMEKPGLKRGGAKGRTLGEVWKERLNHMTKEEFTRYRKEAITEVDRSAAKHARREGNITGGHPVSRGTAKLRWLVERRFLEPVGKVVDLGCGRGGWCYYMATQKRVQEVKGYTKGGPGHEEPQLVQSYGWNIVTMKSGVDVFYRPSEASDTLLCDIGESSSSAEVEEHRTVRVLEMVEDWLHRGPKEFCIKVLCPYMPKVIEKMETLQRRYGGGLIRNPLSRNSTHEMYWVSHASGNIVHSVNMTSQVLLGRMEKKTWKGPQFEEDVNLGSGTRAVGKPLLNSDTSKIKNRIERLKKEYSSTWHQDANHPYRTWNYHGSYEVKPTGSASSLVNGVVRLLSKPWDTITNVTTMAMTDTTPFGQQRVFKEKVDTKAPEPPEGVKYVLNETTNWLWAFLARDKKPRMCSREEFIGKVNSNAALGAMFEEQNQWKNAREAVEDPKFWEMVDEEREAHLRGECNTCIYNMMGKREKKPGEFGKAKGSRAIWFMWLGARFLEFEALGFLNEDHWLGRKNSGGGVEGLGLQKLGYILKEVGTKPGGKVYADDTAGWDTRITKADLENEAKVLELLDGEHRRLARSIIELTYRHKVVKVMRPAADGKTVMDVISREDQRGSGQVVTYALNTFTNLAVQLVRMMEGEGVIGPDDVEKLGKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELIGRARISPGAGWNVRDTACLAKSYAQMWLLLYFHRRDLRLMANAICSAVPANWVPTGRTTWSIHAKGEWMTTEDMLAVWNRVWIEENEWMEDKTPVERWSDVPYSGKREDIWCGSLIGTRTRATWAENIHVAINQVRSVIGEEKYVDYMSSLRRYEDTIVVEDTVL

SEQ ID NO: 31 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 32 - protein, 151 aa, Homo sapiens
MSIPFSNTHYRIPQGFGNLLEGLTREILREQPDNIPAFAAAYFESLLEKREKTNFDPAEWGSKVEDRFYNNHAFEEQEPPEKSDPKQEESQISGKEEETSVTILDSSEEDKEKEEVAAVKIQAAFRGHIAREEAKKMKTNSLQNEEKEENK

SEQ ID NO: 33 - DNA, 52 bp, Artificial Sequence
  SBC-170,005 aptamer
accgtgtagcacatcaacgcatgctncncgttacgatgcatgctgccagcat

SEQ ID NO: 34 - DNA, 52 bp, Artificial Sequence
  SBC-170,009 aptamer
accgtgtagcacatcagataataatccntngacaggtgcatgctgccagcat

SEQ ID NO: 35 - DNA, 51 bp, Artificial Sequence
  SBC-170,013 aptamer
accgtgtagcacatcaccctaacacncnaggatcatgcatgctgccagcat

(19) United States
(12) Patent Application Publication
(10) Pub. No.: US 2016/0000901 A1
(43) Pub. Date: Jan. 7, 2016

(54) Compositions and Methods for the Production of Virus-Like Particles

(71) Applicant: Shifa Biomedical, Malvern, PA (US)

(72) Inventors: Michael N. Blackburn, Malvern, PA (US); Christopher S. Jones, Malvern, PA (US); Nabil Elshourbagy, Malvern, PA (US)

(21) Appl. No.: 14/768,402

(22) PCT Filed: Mar. 4, 2014
(86) PCT No.: PCT/US14/20356

§ 371 Date: Aug. 17, 2015

Related U.S. Application Data
(60) Provisional application No. 61/772,774, filed on Mar. 5, 2013.

(51) Int. Cl. A61K 39/145; C07K 14/005

(52) U.S. Cl. CPC A61K 39/145; C07K 14/005; A61K 2039/6075

(57) ABSTRACT

Compositions and methods for synthesizing virus-like particles (VLPs) and methods of use thereof are provided.

This application claims priority under 35 U.S.C. §119(e) to U.S. Provisional Patent Application No. 61/772,774, filed Mar. 5, 2013. The foregoing application is incorporated by reference herein.

This invention was made with government support under Grant No. 10843353 awarded by the National Institute of Allergy and Infectious Diseases (NIAID). The government has certain rights in the invention.

### FIELD OF THE INVENTION

The present invention relates to the field of virology. Specifically, compositions and methods for synthesizing virus-like particles and methods of use thereof are disclosed.

### BACKGROUND OF THE INVENTION

Several publications and patent documents are cited throughout the specification in order to describe the state of the art to which this invention pertains. Each of these citations is incorporated herein by reference as though set forth in full.

Emerging and re-emerging infectious diseases represent a major risk factor in both the developed and developing worlds and are a significant cause of death and morbidity. Infectious pathogens include prions, viruses, bacteria, fungi, protozoa and multicellular parasites. Before the development of vaccines and anti-infective drugs, infectious diseases were the major cause of death worldwide as recently as the 1940s. Whereas morbidity and mortality data for most diseases such as cancer and cardiovascular diseases are published as a single category, the data for infectious diseases are normally reported for individual illnesses or organisms. For example, the influenza virus is highly infectious and causes both seasonal and pandemic outbreaks of the disease. The number of deaths from seasonal influenza is about 3,000 to about 40,000 per year in the US and 250,000-500,000 per year worldwide. A pandemic outbreak with a highly lethal strain of influenza would result in millions of deaths world-wide. Infections with the highly pathogenic H5N1 avian strains may result in over 50% mortality when they are transmitted to humans.

The prevention and treatment of infectious diseases has taken two paths, treatment of infected individuals with anti-infective drugs and prophylactic prevention of infection with vaccines. Although much progress has been made in the development of anti-infective drugs, vaccines represent the most cost-effective strategy for dealing with these diseases, but the timely design, validation and production of purified vaccines and the supporting analytical reagents are critical challenges that must be resolved for each new infectious disease target. In the case of influenza these issues must be solved every year with the development of new seasonal vaccines. Furthermore, the development of influenza vaccines using the traditional egg-based approach is problematic. For example, the production of egg-based influenza vaccine may take 9-12 months and deliver less than one dose per egg.

A common approach for vaccine development is the use of subunit vaccines, where a surface protein or a fragment of a surface protein is used to elicit an immune response. Over the past decade many new systems for the expression of recombinant subunit influenza viral proteins have been applied to vaccine production to replace the procedures used to make intact but inactivated virus particles. Although the development of recombinant methods for the expression of subunit vaccines has impacted development timelines and accelerated vaccine development, many subunit vaccines do not have the same potency as is observed with the immunization of whole virus particles. This is primarily due to the lower multiplicity of the antigen protein in subunit vaccines when compared to whole virus particles, and that the immune system evolved to respond to antigen presentation in a structurally organized array as seen on the surface of a virus or bacterium. As a consequence, subunit vaccines, which are frequently monomeric or aggregates of variable size, are not as potent as virus particles in eliciting an immune response. One approach to overcome this potency gap has been to design virus-like particles (VLPs) as enveloped particles or as fusions of antigens with the structural or coat proteins of a carrier virus (Crevar et al. (2008) Virology 5:131; Ross et al. (2009) PloS One 4:e6032; Quan et al. (2010) PloS One 5:e9161). Although virus-like-particles or VLPs may bridge this potency gap, the design, expression and purification of VLPs remains problematic and the development of uniform tools to aid in vaccine production is elusive with existing technologies. Not all viral antigens can self-assemble into well-defined particles and the development of cell-based systems to produce VLPs can be both time consuming and costly.

Protein vaccine fusion constructs have been produced in a variety of recombinant systems to generate VLPs. These include bacteria, fungi and plants as well as insect and mammalian cells. These systems overcome some of the obstacles posed by traditional whole virus vaccine production methods. However, the design of these constructs for use in high yield production systems along with the development of high purity and assembly strategies of the assembled VLPs remain as challenges that must be solved for each new vaccine candidate.

Accordingly, it is evident that there is still a strong need for efficient, high yield, and cost effective methods for producing VLPs.

### SUMMARY OF THE INVENTION

In accordance with the present invention, virus-like particles comprising a macromolecular scaffold, at least one multifunctional (inclusive of bifunctional) aptamer, and at least one antigen are provided. In a particular embodiment, the macromolecular scaffold comprises at least one viral capsid or viral capsid component (e.g., from a bacteriophage or a plant virus). In a particular embodiment, the proteins of the macromolecular scaffold and/or the antigen comprise a structural tag (e.g., embedded within the scaffold or antigen structure). The structural tag of the antigen may be the same or different than the structural tag of the scaffold. Compositions comprising at least one virus-like particle and at least one pharmaceutically acceptable carrier are encompassed by the instant invention.

In accordance with another aspect of the instant invention, methods of synthesizing the virus-like particle of the instant invention are provided.

In accordance with an aspect of the instant invention, methods for inhibiting, treating, and/or preventing a disease (e.g., an infectious disease) in a subject are provided.

### BRIEF DESCRIPTIONS OF THE DRAWING

FIG. 1 provides a schematic of certain components of the virus-like particles of the instant invention. Specifically, the scaffold protein (e.g., capsid), the adapter (e.g., bivalent or multivalent aptamers), and the antigen are shown. The bivalent aptamers are depicted linking the antigen to the capsid, whereas monovalent aptamers are not capable. Aptamers may be covalently or non-covalently coupled to the scaffold or to the antigen.

FIG. 2 shows the crystal structure of bacteriophage HK97 gp6 connector protein.

FIGS. 3A-3C provide the amino acid sequences of certain capsid or scaffold proteins.

FIGS. 4A-4C provide the amino acid sequences of certain antigens.

FIG. 5 provides the sequences of certain anti-His tag aptamers. The DNA sequences are provided 5′-3′. The underlined “5” represents dithiol-dT.

### DETAILED DESCRIPTION OF THE INVENTION

The present invention provides a novel technology that allows for the generation of highly potent vaccines using a “plug-and-play cassette system” that can be applied to all vaccines (e.g., antimicrobial, anti-virals, anti-bacterial, etc.) with minimal changes to the system. This invention allows for integrating recombinant proteins into the structure of VLPs using a highly selective bivalent or multivalent cross-linking adapter that cross-links a subunit antigen to a tagged VLP. This novel strategy enables the rapid and effective production of VLPs with a cassette-based tag and tether system based on the use of a genetically encoded protein structural motif, a linker DNA, RNA or peptide nucleic acid (PNA) aptamer or other selective cross-linking technologies and a tagged virus capsid or multimeric protein scaffold. Linking these cassette components in the way described herein represents a novel combinatorial use of these technologies.

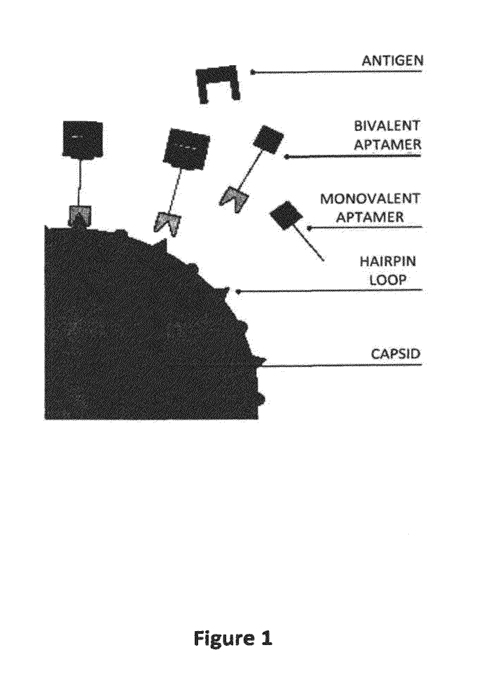

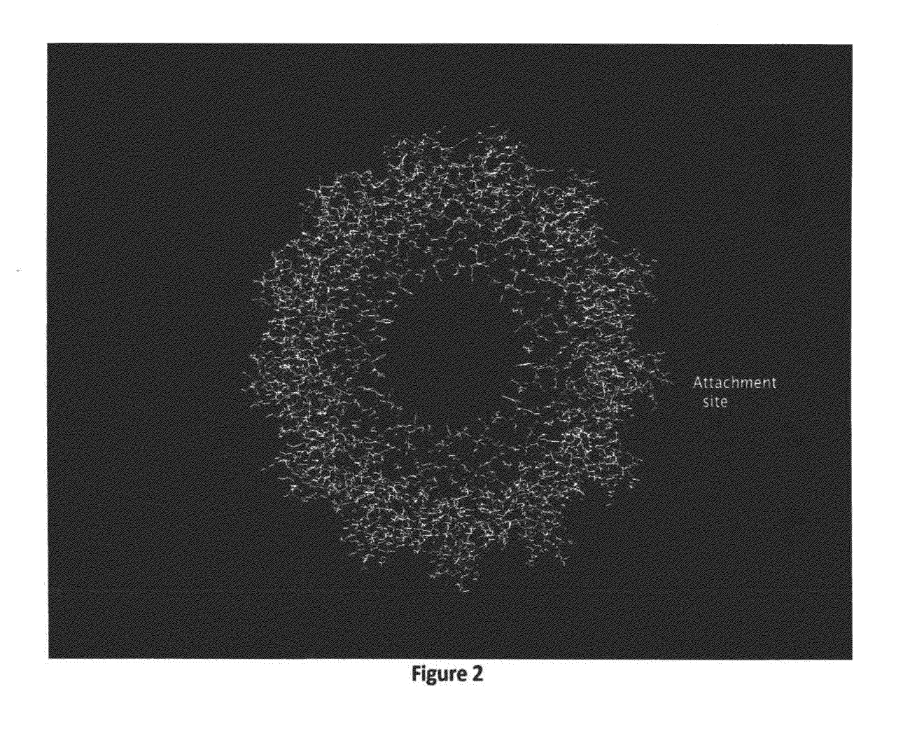

As illustrated in FIG. 1, the system comprises at least one of each of the following cassette components: 1) scaffold protein; 2) adapter; and 3) antigen.

The scaffold protein may be a virus capsid or multimeric protein scaffold composed of multiple copies of one or more proteins. The resultant VLP may, therefore, comprise a structure consisting of a single scaffold protein or a structure comprising more than one different scaffold protein. The capsid/scaffold protein of the instant invention may further comprise at least one structural motif or tag. The structural motif/tag may be a “purification tag,” “affinity tag,” or “epitope tag.” Such tags are well known in the art (see, e.g., Sambrook et al., Molecular Cloning, Cold Spring Harbor Laboratory) and include, but are not limited to: polyhistidine tags (e.g., 4-10 histidines, particularly 6-8 histidines, more typically six histidines), polyarginine tags, glutathione-S-transferase (GST), maltose binding protein (MBP), S-tag, influenza virus HA tag, thioredoxin, staphylococcal protein A tag, the FLAG epitope (DYKDDDDK; SEQ ID NO: 1), AviTag™ epitope (for subsequent biotinylation; GLNDIFEAQKIEWHE; SEQ ID NO: 2), dihydrofolate reductase

(DHFR), an antibody epitope (e.g., a sequence of amino acids recognized and bound by an antibody), the c-myc epitope, a viral nucleotide binding motif, Rev peptide (TRQARRNRRRRWRERQR; SEQ ID NO: 3), TAT peptide (GRKKRRQRRRPQ; SEQ ID NO: 4), zinc-finger motifs/tags, heme binding peptides, and amino acid side-chains that allow selective chemical labeling such as a cysteine thiol. In a particular embodiment, the structural tag comprises amino acids, particularly about 3 to about 100 amino acids or about 4 to about 40 amino acids. In a particular embodiment, the tag is a polyhistidine (e.g., hexa-histidine), zinc-finger tag, or amino acid side-chains that allow selective chemical labeling such as a cysteine thiol.

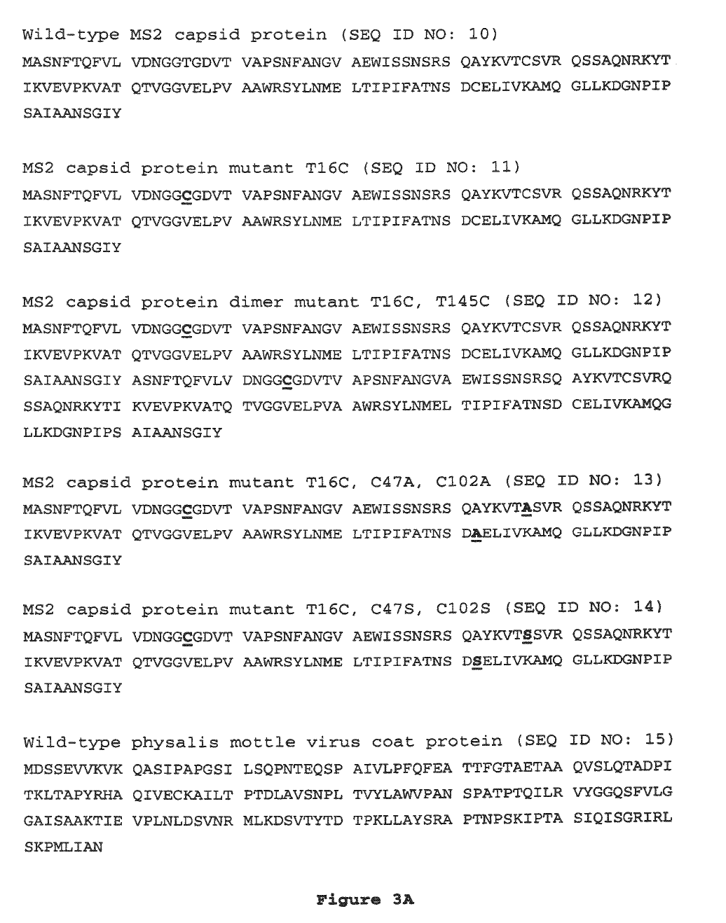

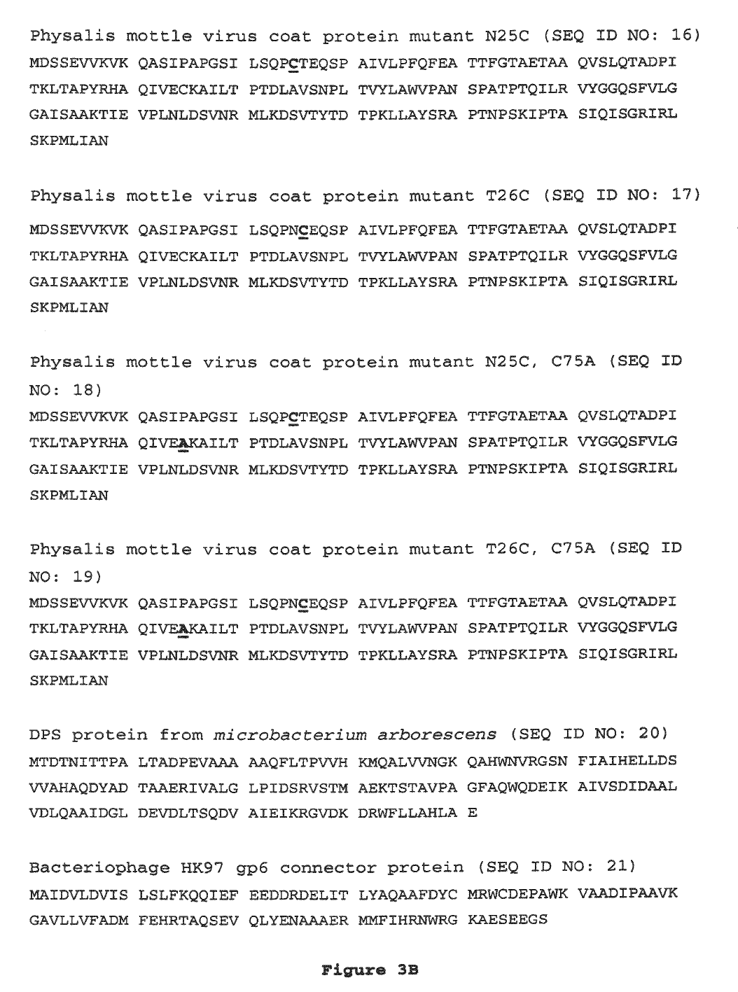

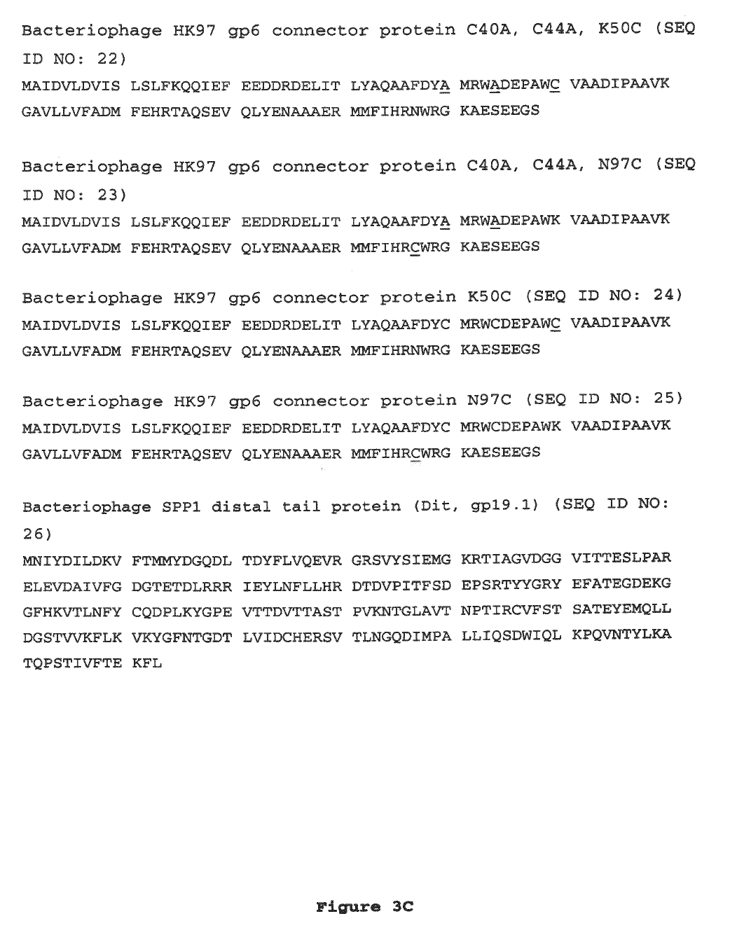

In a particular embodiment, the capsid or scaffold used to generate the VLP used in the practice of this invention is a viral capsid protein that forms icosahedral, dodecahedral, quasi-spherical, filamentous, rod-like, or donut-like structures. In a particular embodiment, the capsid or scaffold protein is from a virus with an icosahedral, quasi-spherical, filamentous, or rod-like structure such as bacteriophage MS2, physalis mottle virus, Ryegrass mottle virus, sobemovirus, Q beta phage, Phi X174 phage, alpha3 phage, alfalfa mosaic virus, tobacco mosaic virus, satellite tobacco necrosis virus, and brome mosaic virus. In a particular embodiment, the capsid or scaffold protein is from a plant virus listed in the Q-bank Plant Viruses and Viroids database (www.q-bank.eu/Virus/). Examples of capsids and scaffolds used to generate the VLPs include, without limitation: wild-type MS2 capsid protein; MS2 capsid protein mutant T16C; MS2 capsid protein dimer mutant T16C, T145C; MS2 capsid protein mutant T16C, C47A, C102A; MS2 capsid protein mutant T16C, C47S, C102S; wild-type physalis mottle virus coat protein; physalis mottle virus coat protein mutant N25C; physalis mottle virus coat protein mutant T26C; physalis mottle virus coat protein mutant N25C, C75A; physalis mottle virus coat protein mutant T26C, C75A; DPS (DNA protection during starvation) protein from microbacterium arborescens (which self-associates to form an oligomeric structure containing 12 highly helical polypeptide chains); bacteriophage HK97 gp6 connector protein (which self-associates to form an oligomeric toroid-like structure); bacteriophage HK97 gp6 connector protein C40A, C44A, K50C; bacteriophage HK97 gp6 connector protein C40A, C44A, N97C; bacteriophage HK97 gp6 connector protein K50C; siphophage SPP 1 distal tail protein (Dit, gp 19.1) (Veesler et al. (2010) J. Biol. Chem., 285:36666-36673); and bacteriophage HK97 gp6 connector protein N97C (see FIG. 3). In a particular embodiment, the capsid/scaffold protein of the instant invention comprises a sequence having at least about 50%, 60%, 70%, 75%, 80%, 85%, 90%, 95%, 97%, 99%, or 100% identity with the sequences provided in FIG. 3.

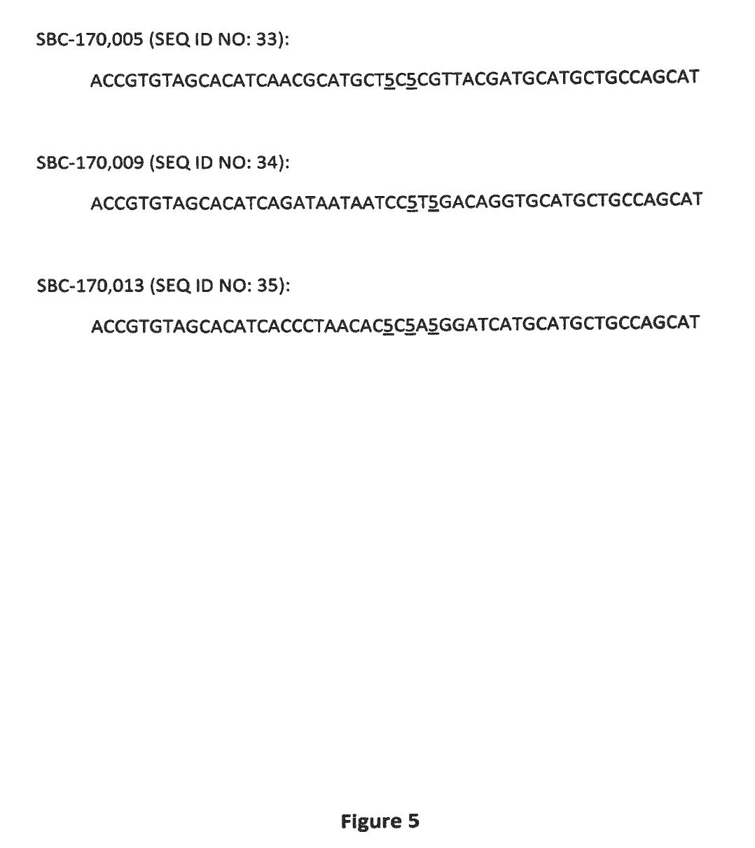

The adapter of the instant invention may be a bivalent or multivalent adapter (e.g., aptamer) that recognizes or specifically binds the capsid/scaffold protein and the antigen or the structural motifs/tag attached thereto. In a particular embodiment, the adapter is a bifunctional or multifunctional cross-linking agent. The tethering adapters thus create a highly structured repeating array for the presentation of antigen to the immune system. Examples of adapters include, but are not limited to: cross-linkers (e.g., chemical cross-linkers), peptides (e.g., short peptides of about 1 to about 10 or 20 amino acids), RNA, DNA, PNA (peptide nucleic acids), and aptamers. PNAs are nucleic acids attached through a peptide backbone sequence. As stated above, the aptamers can be selected to bind to sequences of the capsid/scaffold protein and the antigen or to the added tags. Aptamers have been generated which can bind to the hexa-his tag, Rev peptide (Xu et al. (1996) PNAS 93:7475-7480), TAT peptide (Matsugami et al. (2004) Nucleic Acids Sym., 48:111-112), zinc finger motifs, etc., with high affinity. These aptamers can be modified with a terminal maleimide (WO 1989/006701) or other reactive group to react covalently with free SH groups encoded within the scaffold sequences. Examples of aptamers include, without limitation: an aptamer (e.g., RNA) which specifically binds the hexa-his tag sequence (e.g., Shot47 (Tsuji et al. (2009) Biochem. Biophys. Res. Commun., 386:227-31)) or an aptamer (e.g., DNA) which specifically binds the hexa-his tag sequence (e.g., 6H7 (Aptagen, LLC, Jacobus P A; Kokpinar et al. (2011) Biotech. Bioengr. 108:2371-2379; 5′-GCTATGGGTG GTCTGGTTGG GATTGGCCCC GGGAGCTGGC-3′; SEQ ID NO: 5)). Examples of DNA aptamers containing modified nucleotides include SBC-170,005, SBC-170,009, and SBC-170,013 (see FIG. 5). The aptamer may be modified at either the 5′ or 3′ end with a bifunctional maleimide reagent to allow covalent labeling of free thiols.

The tethering adaptor can also be attached to the scaffold using a duplex nucleic acid pair where a first oligonucleotide chain is linked to the capsid/scaffold and a second oligonucleotide chain (which is complementary to the first) is linked to the antigen. Such duplex binding structures can be formed by base pairing between DNA, RNA or PNA (peptide nucleic acids). In a particular embodiment, the first and second oligonucleotides are complementary (e.g., form a duplex) over a region of about 5 to about 50 nucleotides, particularly about 10 to about 25 nucleotides. The oligonucleotides typically have a length of about 10 to about 250 nucleotides, about 20 to about 200, about 20 to about 100, or about 20 to about 50 nucleotides.

Antigens of the instant invention can be proteins or peptides, nucleic acids, lipids or glycolipids or small molecules (e.g., small organic compounds). The VLPs of the instant invention may comprise one or more different antigens. In a particular embodiment, at least one structural motif or tag (e.g., hexa-his or a zinc finger motif) is attached to the antigen. The at least one structural motif or tag may be the same or different than the one attached to the scaffold protein/capsid. In a particular embodiment, the antigen may be the globular binding domain of the influenza hemagglutinin (HA) or the intact HA chain. However, by swapping different antigens for the HA antigen cassette component, optionally while maintaining the hexa-histidine or zinc-finger tags, new vaccines against a variety of disease targets can be produced without having to re-engineer the entire system. In addition to aptamers against the hexa-histidine or zinc-finger motifs, selective aptamers to other structures can be used to provide a battery of reagents to employ in this “plug-and-play cassette system.”

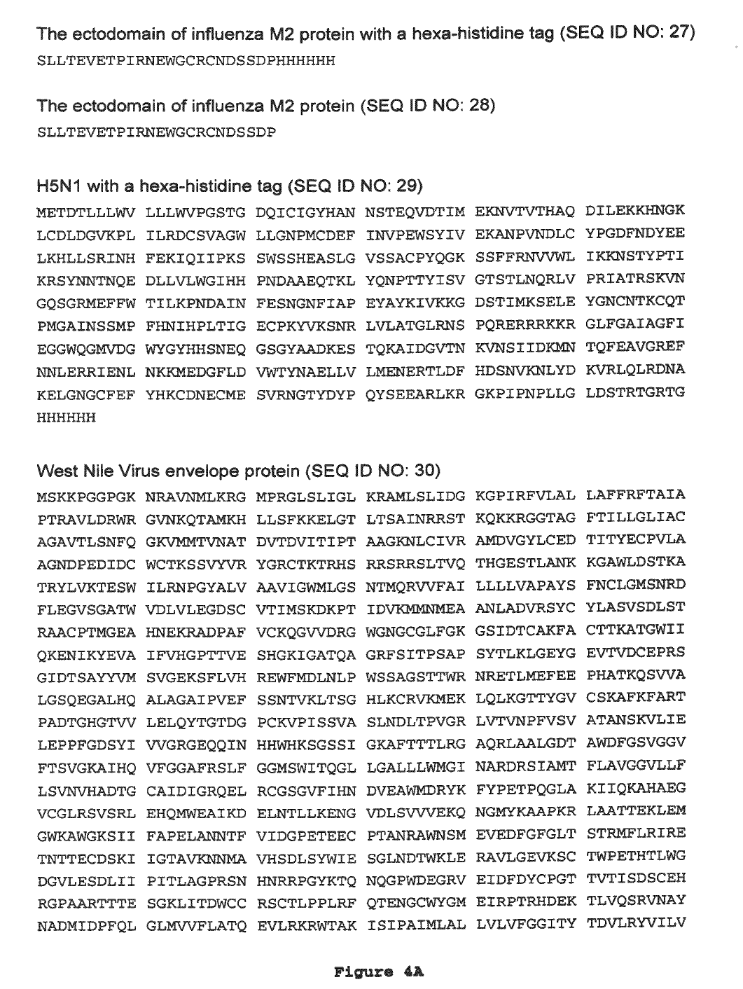

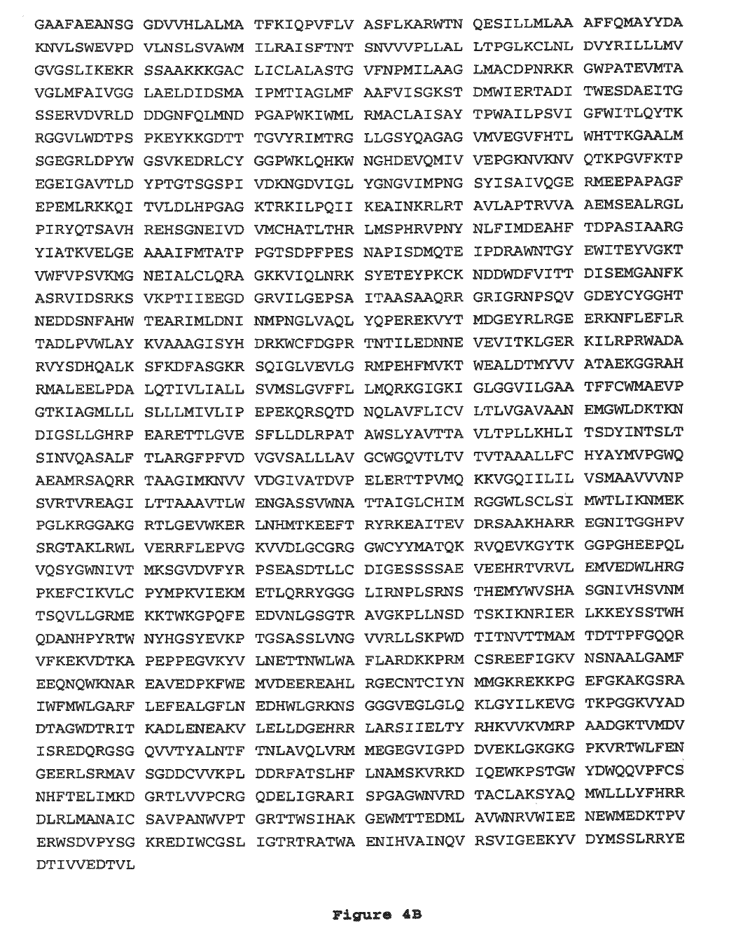

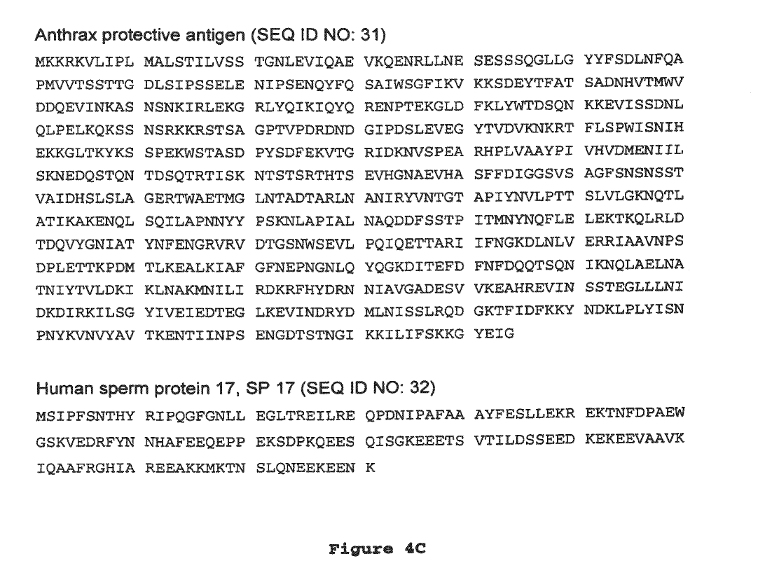

Examples of antigens include, without limitation: hemagglutinin (e.g., the H1N1-HA from the influenza strain A/Mexico/04/2009; or H5N1); the ectodomain of influenza M2 protein, optionally with a hexa-histidine tag; influenza neuraminidase; influenza nuclear protein; West Nile Virus envelope protein or fragment thereof; anthrax protective antigen; bacterial cell surface oligosaccharides including Mycobacterium tuberculosis phosphatidylinositol mannosides, Salmonella polysaccharide, Pneumococcal polysaccharide, etc.; small molecules such as nicotine, heroin or other drugs of abuse; venoms (e.g., from snakes, spiders or insects); toxins (e.g., from plants such as abrin or ricin); cancer-related antigens (e.g., human sperm protein SP 17, human epidermal growth factor receptor 2 (HER2; Gene ID: 2064), mucin1 (MUC1; Gene ID: 4582)), and epitopes thereof (see FIG. 4). In a particular embodiment, the antigen of the instant invention comprises a sequence having at least about 50%, 60%, 70%, 75%, 80%, 85%, 90%, 95%, 97%, 99%, or 100% identity with the sequences provided in FIG. 4. In a particular embodiment, the antigen is a fragment of the full length protein, particularly an epitope.

Methods of synthesizing VLPs are also encompassed by the instant invention. The methods comprise combining the scaffold protein/capsid, adapter, and antigen and isolating (or purifying) the resultant VLPs. In a particular embodiment, the scaffold protein/capsid is assembled into particles (e.g., macromolecular scaffold) and then isolated prior to being contacted with the adapter and antigen.

The instant invention also encompasses compositions comprising at least one VLP and at least one pharmaceutically acceptable carrier. The compositions may further comprise at least one other anti-microbial or vaccine (e.g., against the pathogen or disease to which the VLP is directed).

The instant invention also encompasses methods of inhibiting, treating, and/or preventing a disease or disorder in a subject. The methods comprise administering at least one VLP of the instant invention to the subject. In a particular embodiment, the method comprises administering the VLP in a composition with at least one pharmaceutically acceptable carrier. In a particular embodiment, the method comprises inhibiting, treating, and/or preventing an infectious disease, particularly the prevention of the infectious diseases (e.g., administering the VLP as a vaccine). The methods of the instant invention can be co-administered (sequentially and/or simultaneously) with at least one other therapeutic and/or adjuvant for the treatment and/or prevention of the disease.

The compositions of the present invention can be administered by any suitable route, for example, by injection (e.g., for local, direct, or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can be prepared, for example, in liquid form, or can be in dried powder form (e.g., lyophilized for later reconstitution).

The therapeutic agents described herein will generally be administered to a patient as a pharmaceutical preparation. The term “patient” as used herein refers to human or animal subjects. The compositions of the instant invention may be employed therapeutically or prophylactically, under the guidance of a physician.

The compositions comprising the agent of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated.

The dose and dosage regimen of the agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular condition may be determined by dosage concentration curve calculations, as known in the art.

The pharmaceutical preparation comprising the agent may be administered at appropriate intervals, for example, 7 to 28 day intervals or as appropriate to achieve the desired immune response.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### Definitions

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); German), A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., an infectious disease) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate stress and/or stress response in a subject.

As used herein, the term “subject” refers to an animal, particularly a mammal, particularly a human.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” may refer to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

The term “crosslinker” refers to a molecule capable of forming a covalent or non-covalent linkage between two compounds. Typically, at least part of the crosslinker forms a part of the linkage between the conjugated molecules after the reaction. In a particular embodiment, the crosslinker forms a covalent linkage.

The term “aptamer” refers to a molecule (e.g., a nucleic acid molecule) that specifically binds to a particular molecule of interest or a target, particularly with high affinity and specificity. The aptamer is typically a nucleic acid molecule that has been specifically engineered or selected to bind to a target molecule (see, e.g., Brody et al. (2000) J. Biotechnol., 74:5-13; Leary, J. F. (2005) 5692:216-223; Yang et al. (2008) 183:469-472; Yang et al. (2004) Curr. Drug Targets, 5:705-715). The aptamers may be generated through repeated rounds of in vitro selection or SELEX (systematic evolution of ligands by exponential enrichment). The aptamer may comprise deoxyribonucleotide and/or ribonucleotides. Aptamers are typically single stranded. The aptamers may contain modifications, e.g. non-natural or modified nucleotides such as 2′-substituted (e.g., 2′-fluoro) nucleotides and/or modified backbones such as PNAs. Aptamers are typically about 10 to about 100 nucleotides in length, about 15 to about 75 nucleotides, about 20 to about 60 nucleotides, about 25 to about 50 nucleotides, or about 30 to about 45 nucleotides.

As used herein, the term “virus-like particle” refers to a structure resembling a virus particle but which is non-pathogenic, non-replicative, and non-infectious as it lacks all or part of the viral genome.

The term “specifically binds” refers to a molecule that binds to one or more epitopes of a protein or compound of interest, but which does not substantially recognize and bind other molecules in a sample containing a mixed population of biological molecules.

The following examples provide illustrative methods of practicing the instant invention and are not intended to limit the scope of the invention in any way.

### EXAMPLE 1

In this example, the VLP capsid is formed from MS2 T16C protein, while the adapter is an aptamer and the antigen is the influenza hemagglutinin protein. Influenza constructs are commonly expressed with hexa-his tags to facilitate purification. The hexa-his tag also functions as a recognition site for the anti-his aptamer. A second aptamer recognition site such as the zinc-finger domain sequence from the influenza virus M1 protein can be used as an alternative strategy. An example of the zinc finger motif to be used is a 28 amino acid sequence corresponding to residues 139-166 of the M1 protein (A/California/07/2009(H1N1)) TTEAAFGLVCATCEQIADSQ HRSHRQMA (SEQ ID NO: 6). This is a Cys2-His2 zinc finger. This zinc finger domain peptide is known to bind 1 mole of zinc or cobalt and undergoes a metal-dependent change in conformation which involves the stabilization of helical structures in the peptide (Hui et al. (2006) J. Virol., 80:5697-707; Hui et al. (2003) J. Gen. Virol., 84:3105-3113; Okada et al. (2003) Biochem., 42:1978-1984). Similar results have been observed with other zinc finger domains that contain alpha helix structures (Frenkel et al. (1987) Proc. Natl. Acad. Sci., 84:4841-4845). Biophysical studies suggest that the zinc-binding residues are flanked by two helices. This is consistent with the x-ray structure of M1 (Arzt et al. (2001) Virol., 279:439-446), where the zinc finger motif spans the two domains of the matrix protein. Use of virally derived protein tags may enhance immunogenicity while minimizing the potential of developing immune responses that might be generated against protein tag sequences derived from non-viral origin proteins. The anti-his aptamer was developed as a tool for protein purification. The zinc-finger domain/aptamer pair may also function in this capacity providing a second metal-dependent purification handle for the proteins.

The MS2-T16C mutant protein, with a Cys residue at position 16 within the hairpin loop may be expressed and purified. Previous studies have shown that this construct expresses well in E. coli and assembles into capsids (Peabody D. S. (2003) J. Nanobiotechnol., 1:5-12). Purification of the capsid does require care to prevent disulfide cross-linking and aggregation of capsids but addition of reducing agents prevents this oxidation reaction. Once the Cys16 thiol is reacted with the maleimide-linked aptamer it no longer undergoes oxidation. Notably, there are no other available thiols on the surface of the MS2 virus. The MS2 capsid protein mutants T16C, C47S, C102S or T16C, C47A, C102A are constructed to remove any buried thiols in the capsid.

Two cDNA constructs of the enterobacteriophage MS2 cDNA will be made; wild-type MS2, to be used as a control, and the Cys16 version, MS2-T16C. The cDNA sequences may be obtained by back translation of the open reading frame and are optimized for E. coli expression. Synthesis of the cDNA sequence may be made in pGA18, and then cloned in the pJ expression 404 vector. The two constructs can be verified by sequence analysis. For expression, the two constructs, in BL21 strain of E. coli, may be scaled up under culture conditions of 37° C., pH 7.0, and 20% dissolved oxygen. Protein production may be induced with IPTG (1 mM) during early exponential growth phase (0.6-0.8 OD), and cultures may be extended for 8 additional hours at 30° C. Following lysis of the cell, the MS2 capsids from wild-type and MS2-CT16C may be purified (Peabody D. S. (2003) J. Nanobiotechnol., 1:5-12).

Hemagglutinins may be produced in E. coli using the expression system consisting of a hexa-his tag followed by the enterokinase (EK) cleavage sequence (DDDDK; SEQ ID NO: 7) that is fused to the HA molecule (residues 63-286) (A/Mexico/04/2009(H1N1)). The zinc-finger motif may be inserted between the enterokinase sequence and the start of the hemagglutinin. Two cDNA constructs of the hemagglutinin cDNA (HA63-286) may be made; one without and the other with the 28 AA zinc finger domain. The zinc finger domain may be inserted in frame between EKCS and the H1N1-HA domains. The two constructs may contain the hexa-his tag at the N-terminus followed by an enterokinase recognition site to allow the removal of the histidine tag, if desired. The cDNA sequence may be obtained by back translation of the open reading frame and optimized for E. coli expression. Synthesis of the cDNA sequence may be made in pGA18, and then cloned into a vector such as the pJ expression 404 vector. The two constructs may be verified by sequence, and expressed as described above.

The hemagglutinin constructs may be purified using described procedures (DuBois et al. (2011) J. Virol., 85:865-872; Aguilar-Yanez et al. (2010) PLoS One, 5:e11694), in which unfolded his-tagged hemagglutinin is captured using immobilized metal affinity chromatography followed by refolding of the matrix-bound protein prior to elution with imidazole or other chelators. The zinc finger domain interaction with a selected anti-zinc-finger aptamer provides additional affinity purification options. Ion exchange, gel filtration and hydrophobic interaction chromatography can also be employed. Purity may be assayed by SDS-PAGE.

The purified MS2 capsid and hemagglutinin constructs may be characterized by biophysical and immunologic procedures. The integrity and homogeneity of the capsids may be assessed by size-exclusion chromatography, light scattering or analytical ultracentrifugation. Folding of the hemagglutinin constructs may be determined by CD spectra, or fluorescence melting of the protein. Also, ELISA or BIAcore assays may be used to quantitate interaction of the recombinant hemagglutinins with conformation-dependent anti-hemagglutinin monoclonal antibodies that are available from commercial sources. Controls may include commercially available hemagglutinins.

### EXAMPLE 2

This example will focus on the identification and preparation of aptamers as an example of adapters. Bead-based random oligonucleotide libraries have been used to rapidly identify thioaptamers (Yang et al. (2008) Phosphorus, Sulfur, and Silicon and the Related Elements, 183:469-472). The microbead selection approach uses differential binding of proteins, where the binding of a protein with a specific tag, for example, the M1 zinc finger domain-hemagglutinin fusion, in the presence of competing levels of the same protein, the H1N1 HA, lacking the zinc finger tag. This allows selection of tag-specific aptamers.

Aptamers that bind to the target may be selected using a bead-based approach as outlined by Yang et al. A random DNA oligonucleotide library may be synthesized on beads using a pool and split approach. With this method, each bead will contain about 10¹² copies of a single oligonucleotide sequence of about 30-40 to nucleotides. The oligonucleotides may also contain a defined primer sequence for later PCR sequencing of the selected beads. The library may contain about 20-30% phosphorodithioate nucleosides which add to aptamer stability and to the potential for novel molecular interactions between aptamer and target, thereby increasing both affinity and selectivity. Purified hemagglutinin protein may be biotinylated to achieve an average labeling of about 1.5 biotin moieties per polypeptide chain. The beads may be mixed with sub-nanomolar concentrations of the biotinylated target protein in the presence of a large excess of non-tagged protein and beads containing selectively bound target protein may be captured using streptavidin-coated magnetic particles. Individual beads that are selected by this system may be PCR amplified and sequenced.

The sequenced aptamers that are selected in this bead-selection round may be re-synthesized and specific binding confirmed using mobility shift assays or by ELISA. These assays will also provide preliminary affinity binding data.

The bifunctional aptamers containing the thiol reactive maleimide may be designed with a poly-A tail for attachment of the maleimide. Studies have used this approach to link oligonucleotides to peptide or protein thiol groups (Tung et al. (1991) Bioconjugate Chem., 2:464-465).

The interaction of the aptamers with their target proteins may be characterized using biophysical and immunologic techniques. The affinity, binding kinetics and stoichiometry of the binding interactions may also be determined. The interaction of the hemagglutinin constructs may be measured independently for the MS2 capsid-aptamer complex and for the monomeric aptamer to demonstrate that hemagglutinin binds to aptamer and to aptamer-capsid complex with similar affinities. Binding kinetics and affinity are measured by surface plasmon resonance using a BIAcore 3000. Protein may be immobilized onto chips using standard coupling chemistry. Coupling density may be selected to minimize mass transport and rebinding effects. Data may be analyzed by non-linear regression to obtain association and dissociation rate constants. Stoichiometry and affinity may be measured by isothermal titration calorimetry (ITC) using a Microcal ITC. The titration data may be analyzed using Origin software to obtain stoichiometry and KD. Stoichiometry of interaction between MS2-aptamer complex and the HA construct may also be determined by sedimentation velocity titration experiments in a Beckman XLI analytical ultracentrifuge using either absorbance or interference optics (Doyle et al. (2000) Meth. Enzymol., 323:207-230). Interaction of anti-hemagglutinin antibodies may also be analyzed for the complex and for the monomeric hemagglutinins by ELISA and BIAcore.

Anti-His tag aptamers were identified using the above described procedure by selective binding of the biotinylated peptide, biotin-GDSTRTGRTGHHHHHH (SEQ ID NO: 8), which includes the C-terminal hexa-His sequence. Binding of the three high-affinity aptamers (SBC-170,005, SBC-170,009, SBC-170,013) to hexa-His labeled peptide of proteins was characterized by biosensor analysis using a ForteBio Octet® system, yielding dissociation constants of 50 to 150 nM. SBC-170,013 containing a 5′ maleimide was synthesized by solid phase methods and linked to purified MS2-T16C scaffold via the cysteine residue at position 16 in the MS2 protein.

### EXAMPLE 3

MS2-T16C—mal-6H7-anti-His aptamer—H5N1 HA-hexa-His

The scaffold, MS2-T16C, may be purified and labeled with the anti-hexa-histidine aptamer 6H7 by coupling the free reactive thiols of MS2-T16C to a maleimide moiety at the 5′ terminus of 6H7. The anti-hexa-histidine aptamer may be used to capture the influenza hemagglutinin antigen H5N1-hexa-his which may be expressed using 293-cells and purified from the media using metal-chelate chromatography.

MS2-T16C—mal-6H7-anti-His aptamer—H1N1 HA-hexa-His

The H5N1 His-tagged HA can be replaced with a His-tagged H1N1 and then expressed, refolded and purified using an E. coli expression system.

MS2-T16C—mal-6H7-anti-His aptamer—M2e-hexa-His

The scaffold, MS2-T16C, may be purified and labeled with the anti-hexa-histidine aptamer 6H7 by coupling the free reactive thiols of MS2-T16C to a maleimide moiety at the 5′ terminus of 6H7. The anti-hexa-histidine aptamer may be used to capture the influenza M2 extracellular domain, M2e, via a fused hexa-histidine tag. M2e (SLLTEVETPIRNEWGCRCNDSSDPHHHHHH; SEQ ID NO: 9) can be prepared by solid-state peptide synthesis.

MS2-T16C—mal-6H7-anti-His aptamer—Hexa-his Tagged Cancer Related Antigen Sperm Protein, SP 17

The scaffold, MS2-T16C, is purified and labeled with the anti-hexa-histidine aptamer 6H7 by coupling the free reactive thiols of MS2-T16C to a maleimide moiety at the 5′ terminus of 6H7. The anti-hexa-histidine aptamer is used to capture the SP 17 protein with a fused N-terminal hexa-his tag.

The above aptamers 6H7 can be replaced with other anti-His aptamers such as Shot 47, SBC-170,005, SBC-170,009, or SBC-170,013. The above antigens can also be replaced with other antigens such as West Nile Virus envelope glycoprotein-H₆ or anthrax protective antigen-H₆. Further, as explained hereinabove, the scaffold proteins do not need to be viral capsid proteins as other oligomeric proteins can be used. The MS2 capsid protein may be replaced with the DPS protein from microbacterium arborescens or the connector protein gp6 from bacteriophage HK97. For example, the VLP may comprise HK97 gp6 K50C—mal-Aptamer Shot 47—influenza M2e-H₆ or HK97 gp6 N97C—mal-Aptamer 6H7—influenza H5N1 HA-H₆. Notably, by preparing the hybrid HK97 gp6 oligomer with both of these constructs would yield a particle carrying both the HA antigen and the Me2 antigen.

While certain embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of producing virus-like particles, said method comprising linking at least one antigen to a macromolecular scaffold with a multifunctional adapter.

2. The method of claim 1, where the macromolecular scaffold comprises at least one viral capsid or viral capsid component.

3. The method of claim 2, wherein said viral capsid is from a bacteriophage.

4. The method of claim 3, wherein said bacteriophage is selected from the group consisting of MS2, Qbeta, and PhiX174.

5. The method of claim 2, wherein said viral capsid is from a plant virus.

6. The method of claim 5, wherein said plant virus is selected from the group consisting of the Physalis mottle virus, alfalfa mosaic virus, satellite tobacco necrosis virus and tobacco mosaic virus.

7. The method of claim 6, wherein said plant virus is the Physalis mottle virus.

8. The method of claim 1, wherein said macromolecular scaffold and/or antigen comprises a structural tag.

9. The method of claim 8, wherein said adapter specifically binds said structural tag.

10. The method of claim 8, wherein said structural tag comprises about 4 to about 40 amino acid residues.

11. The method of claim 10, wherein said structural tag comprises 4 to 10 histidine residues.

12. The method of claim 8, wherein said structural tag is a zinc finger motif.

13. The method of claim 8, wherein said structural tag the Rev peptide or the Tat peptide.

14. The method of claim 1, wherein said adapter is a nucleic acid aptamer.

15. The method of claim 14, wherein said aptamer is coupled to the scaffold and/or the antigen by a cysteine thiol moiety.

16. The method of claim 14, wherein said aptamer comprises of two distinct hybridized monofunctional aptamers.

17. The method of claim 16, wherein the two distinct aptamers bind different protein sequences or structural tags.

18. The method of claim 1, wherein the scaffold comprises a virus structural component.

19. The method of claim 17, wherein said virus structural component is the bacteriophage HK97 gp6 connector protein.

20. The method of claim 1, where the scaffold comprises Ryegrass mottle virus coat protein or other sobemovirus capsids.

21. The method of claim 1, wherein the scaffold comprises proteins having at least one cysteine substitution mutation.

22. A virus-like particle comprising a macromolecular scaffold, at least one antigen, and at least one multifunctional adapter, wherein said adapter links said antigen to said macromolecular scaffold.

23. A composition comprising at least one virus-like particle of claim 22 and at least one pharmaceutically acceptable carrier.

24. A method for preventing or treating a disease in a subject, said method comprising administering to said subject at least one virus-like particle of claim 21, optionally with at least one pharmaceutically acceptable carrier, to said subject.
